(19) United States
(12) Patent Application Publication
(10) Pub. No.: US 2004/0254154 A1
(43) Pub. Date: Dec. 16, 2004

(54) Prediction of changes to visual acuity from assessment of macular edema

(75) Inventor: Paul Ashton, Boston, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA

(21) Appl. No.: 10/841,608

(22) Filed: May 7, 2004

Related U.S. Application Data
(60) Provisional application No. 60/468,964, filed on May 7, 2003.

(51) Int. Cl. A61K 31/573

(52) U.S. Cl. 514/179

(57) ABSTRACT

The instant invention provides methods (including business methods) and reagents (including packaged pharmaceutical compositions) for use in predicting the long term effect on visual acuity (VA) of a pharmaceutical or treatment regimen in a patient with macular edema.

### RELATED APPLICATION

This application claims the benefit of U.S. Provisional Application No. 60/468,964, filed May 7, 2003, the specification of which is hereby incorporated by reference in its entirety.

### BACKGROUND OF THE INVENTION

The need to shorten duration and cost of clinical trials has stimulated interest in the development of biomarkers and surrogate endpoints that may substitute for clinical endpoints. The treatment of surrogate endpoints in the Medical and Statistics literature has often been heuristic and ad hoc in character. For instance, an inherent limitation of current surrogate endpoint validation techniques is its general failure in predicting outcome in treating diseases which are multifactorial in terms of the physiological and/or behavioral changes that may occur in populations suffering from the disease.

There is currently a need for more practical techniques for using surrogate endpoints to supplement standard analysis on final endpoints.

Macular edema occurs when as a result of fluid accumulation around the macula—the most sensitive portion of the retina that is crucial for seeing fine detail—severe blurring of vision may occur. A number of ophthalmic diseases lead to macular edema, such as diabetic macular edema (DME).

### SUMMARY OF THE INVENTION

One aspect of the invention provides a packaged pharmaceutical comprising: (A) a pharmaceutical formulation including one or more drugs that may affect visual acuity; (B) instructions for assessing a patient to whom said pharmaceutical formulation is administered and who presents some degree of macular edema, said instructions providing for altering dosage regimen and/or discontinuing administration if the degree of macular edema does not decrease after administration of said formulation, changes in said degree of macular edema being predictive for long term changes in visual acuity.

In one embodiment, said pharmaceutical formulation is a sustained-release formulation.

In one embodiment, said pharmaceutical formulation is provided in a sustained-release device.

In one embodiment, said pharmaceutical formulation is for treating an ophthalmic disorder.

In one embodiment, said formulation is delivered by Vitrasert® implant, Envision TD™ or Posurdex™.

In one embodiment, said formulation is delivered using a device using AEON™ technology or CODRUG™ technology.

In one embodiment, said ophthalmic disorder is: posterior uveitis, Diabetic Macular Edema (DME), Wet ARMD, or CMV retinitis.

In one embodiment, said pharmaceutical formulation is for intraocular injection or implantation.

In one embodiment, said pharmaceutical formulation comprises one or more of an anti-inflammatory compound, neuroprotective agent, and/or immunomodulatory compounds (such as cyclosporin A or FK506, etc.).

In one embodiment, said assessment of severity of said edema is effectuated by directly measuring macular edema.

In one embodiment, said measuring of macular edema includes measuring the area, volume, thickness (height or elevation) of said edema.

In one embodiment, the measurement of macular edema is directly measured by infrared scanning laser tomography or optical coherence tomography (OCT).

In one embodiment, said assessment of severity of said edema is effectuated by comparing a diseased macular edema with a normal macular, followed by grading the severity of edema.

In one embodiment, said pharmaceutical formulation includes a corticosteroid. For example, said corticosteroid is: triamcinolone, dexamethasone, fluocinolone, cortisone, prednisolone, flumetholone, or derivatives thereof. Preferably, said corticosteroid is triamcinolone acetonide (TA) or fluocinolone acetonide (FA).

In one embodiment, said instructions for assessing the patient include instructions to measure the area, volume, thickness (height or elevation) of the macular edema.

In one embodiment, said instructions set forth clearance of edema as being predictive of lower percentage of patients with greater than or equal to a 15 letter loss in visual acuity.

In one embodiment, said sustained-release formulation is capable of being released over a period of about 1 month to about 20 years, preferably over a period of about 6 months to about 5 years.

In one embodiment, said instructions include monitoring the degree of macular edema in said patient for about 2-18 months, preferably 6-12 months.

In one embodiment, the sustained release device is a biocompatible implantable ocular controlled release drug delivery device sized for implantation within an eye for continuously delivering said pharmaceutical formulation within the eye for a period of at least several weeks, which device comprises a polymeric outer layer that is substantially impermeable to the drug and ocular fluids covering a core comprising pharmaceutical formulation, wherein said outer layer has one or more orifices that create a flow path through which fluids may pass to contact the core and dissolved drug may pass to the exterior of the device.

In one embodiment, the device further includes one or more semi-permeable layers disposed in said flow path, which semi-permeable layers are at least partially permeable to dissolved drug, wherein said semi-permeable layers reduce influx of proteins from ocular fluid and/or reduce the rate of release of dissolved drug from the device.

In one embodiment, the rate of release of drug is determined solely by the composition of the core and the total surface area of the one or more orifices relative to the total surface area of said device.

In one embodiment, said outer layer comprises polytetrafluoroethylene, polyfluorinated ethylenepropylene, polylactic acid, polyglycolic acid, or silicone or a mixture thereof.

In one embodiment, the outer layer is biodegradable.

In one embodiment, said semipermeable layer comprises PVA (poly(vinyl acetate)).

In one embodiment, the sustained release formulation is a biodegradable implant comprising said one or more drugs and a biodegradable polymer.

In one embodiment, said one or more drugs comprise about 50-80 weight percent of the implant.

It should be understood that all embodiments described above may be combined with one or more of other embodiments when appropriate.

Another aspect of the invention provides a method for assessing the long term effect on visual acuity (VA) of a pharmaceutical formulation for treatment in a patient who presents some degree of macular edema, the method comprising assessing degree of macular edema before and after said treatment, wherein a reduction in said severity is predictive of increased long term benefit of improvement in visual acuity, and/or decreased long term risk of deterioration in visual acuity.

In one embodiment, said pharmaceutical formulation is a sustained-release formulation.

In one embodiment, said pharmaceutical formulation is provided in a sustained-release device.

In one embodiment, said pharmaceutical formulation is for treating an ophthalmic disorder.

In one embodiment, said ophthalmic disorder is: posterior uveitis, Diabetic Macular Edema (DME), Wet ARMD, or CMV retinitis.

In one embodiment, said treatment is directed to a condition unrelated to an ophthalmic disorder, and wherein said effect is a side effect of said treatment.

In one embodiment, said pharmaceutical formulation is for intraocular injection or implantation.

In one embodiment, said pharmaceutical formulation comprises one or more of an anti-inflammatory compound, neuroprotective agent, and/or immunomodulatory compounds.

In one embodiment, said pharmaceutical formulation includes a corticosteroid.

In one embodiment, said corticosteroid is: triamcinolone, dexamethasone, fluocinolone, cortisone, prednisolone, flumetholone, or derivatives thereof. Preferably, said corticosteroid is triamcinolone acetonide (TA) or fluocinolone acetonide (FA).

In one embodiment, said instructions for assessing the patient include instructions to measure the area, volume, thickness (height or elevation) of the macular edema.

In one embodiment, clearance of edema after said treatment is predictive of lower percentage of patients with greater than or equal to a 15 letter loss in visual acuity.

In one embodiment, the sustained release device is a biocompatible implantable ocular controlled release drug delivery device sized for implantation within an eye for continuously delivering said pharmaceutical formulation within the eye for a period of at least several weeks, which device comprises a polymeric outer layer that is substantially impermeable to the drug and ocular fluids covering a core comprising pharmaceutical formulation, wherein said outer layer has one or more orifices that create a flow path through which fluids may pass to contact the core and dissolved drug may pass to the exterior of the device.

In one embodiment, the device further includes one or more semi-permeable layers disposed in said flow path, which semi-permeable layers are at least partially permeable to dissolved drug, wherein said semi-permeable layers reduce influx of proteins from ocular fluid and/or reduce the rate of release of dissolved drug from the device.

In one embodiment, the rate of release of drug is determined solely by the composition of the core and the total surface area of the one or more orifices relative to the total surface area of said device.

In one embodiment, said outer layer comprises polytetrafluoroethylene, polyfluorinated ethylenepropylene, polylactic acid, polyglycolic acid, or silicone or a mixture thereof.

In one embodiment, the outer layer is biodegradable.

In one embodiment, said semipermeable layer comprises PVA.

In one embodiment, the sustained release formulation is a biodegradable implant comprising said one or more drugs and a biodegradable polymer.

In one embodiment, said one or more drugs comprise about 50-80 weight percent of the implant.

It should be understood that all embodiments described above may be combined with one or more of other embodiments when appropriate.

Another aspect of the invention provides a method for conducting a drug discovery business, comprising: (A) obtaining data measuring severity of macular edema in one or more patients before and after treatment with a test compound; (B) determining, based on the data obtained in (A), whether said severity of macular edema is reduced in said patients after treatment with said test compound; (C) determining the suitability of further clinical development of a test compound which reduces said severity; (D) for compounds selected for further clinical development, conducting therapeutic profiling of the test compound, or analogs thereof, for efficacy and toxicity in animals; and (E) identifying a pharmaceutical preparation including one or more compounds identified in (D) as having an acceptable therapeutic and/or toxicity profile.

In one embodiment, the method further comprises licensing said compounds to a manufacturer for manufacture and sale of a pharmaceutical preparation comprising said compound.

Another aspect of the invention provides a method of marketing a treatment for an ophthalmic disorder, comprising: (A) marketing, to healthcare providers, a pharmaceutical formulation for long-term treatment of said ophthalmic disorder, which formulation includes one or more drugs that mat affect visual acuity when administered over a sustained period of time; and, (B) providing to said healthcare providers instructions for administering said formulation, which instructions include assessing a patient's prognosis with respect to long-term visual acuity by measuring changes, if any, of macular edema as a prediction of visual acuity.

In one embodiment, said pharmaceutical formulation is for intraocular injection or implantation.

In one embodiment, said pharmaceutical formulation is a sustained-release formulation.

In one embodiment, said pharmaceutical formulation is provided in a sustained-release device.

In one embodiment, said ophthalmic disorder is: posterior uveitis, Diabetic Macular Edema (DME), Wet ARMD, or CMV retinitis.

In one embodiment, said pharmaceutical formulation comprises one or more of an anti-inflammatory compound, neuroprotective agent, and/or immunomodulatory compounds.

In one embodiment, said pharmaceutical formulation a corticosteroid.

In one embodiment, said corticosteroid is: triamcinolone, dexamethasone, fluocinolone, cortisone, prednisolone, flumetholone, or derivatives thereof.

In one embodiment, said corticosteroid is triamcinolone acetonide (TA) or fluocinolone acetonide (FA).

It should be understood that all embodiments described above may be combined with one or more of other embodiments when appropriate.

### DETAILED DESCRIPTION OF THE INVENTION

I. Overview

The invention provides methods, reagents and apparatus for predicting the ability or effectiveness of a drug or combination of drugs to bring about a clinically relevant long term result (such as enhanced probability of improving visual acuity, and/or decreased probability of losing visual acuity). In general, the method is based on assessing the ability of a treatment regimen to achieve one or more surrogate endpoints predicted from multivariate analysis of behavioral, biochemical and/or physiological data, such as clinical trial data obtained from a publicly available database. The ability to achieve this endpoint(s) is predictive of the long term outcome of the disease prognosis. In particular, the subject methods and systems can be used to predict the clinical long term outcome for a program of treatment, such as part of a clinical or pre-clinical trial, or as part of a treatment regimen (e.g., to assess if a patient is responsive to a particular treatment, titrate dosages, etc.). The subject methods and systems can also be used in a drug discovery program, e.g., to identify compounds which are likely to be useful in treating a particular condition to achieve a long term outcome, based on their ability to achieve one or more surrogate endpoints in a test animal system or patient data. The present invention also contemplates the use of the subject methods and systems to categorize drugs in terms of their use for achieving a long term outcome of the treatment, based on their surrogate endpoint “signatures”, and additionally contemplates that such signatures can be stored in databases for comparison with other drugs or test compounds. Still another contemplated use of the subject method is in the development or optimization of drug formulations, e.g., that require a particular biodistribution, release profile or other pharmacokinetic parameter.

A salient feature of the subject method is that it uses established surrogate end-points for multifactorial disease. A surrogate endpoint is a laboratory measurement or a physical sign used as a substitute for a clinically meaningful endpoint that measures directly how a patient feels, functions or survives. Changes induced by a therapy on a surrogate endpoint are expected to reflect changes in a clinically meaningful endpoint. Many diseases involve multiple symptoms, the alleviation of which can, if definitively linked to the disease outcome, be used as a basis for selecting a drug candidate, obtaining regulatory (FDA) approval, and/or assessing and modifying treatment regimens for individual patients.

For example, by analyzing publicly available databases, by utilizing, for example, multi-dimensional analysis described below, Applicants have established that the surrogate endpoint of macular edema clearance is found to be a reliable prediction for long term outcome in visual acuity of patients. Such analysis methods may also be useful to identify other surrogate endpoints that may be similarly useful for predicting long term outcomes in other related or unrelated diseases.

Such classification techniques and/or association techniques establish a predictive relationship for disease treatment based on two or more independent factors which can be (readily) measured in the treated patients. Using combinations of machine learning, statistical analysis, modeling techniques and database technology, the method advantageously utilizes data mining techniques to find and identify patterns and relationships in patient data that permits inference of rules for the prediction of drug effects. Such surrogate endpoints can include, and be derived from analysis of biochemical, physiological and/or behavioral changes, including changes that manifest at the level of gross anatomical changes or as changes in cellular (gene expression or other phenotypic or genotypic changes) or metabolic profiles.

The present invention also contemplates methods of conducting informatics and drug assessment businesses utilizing the apparatus, methods and databases of the present invention.

II. Definitions

The term “classification” refers to the problem of predicting the number of sets to which an item belongs by building a model based on some predictor variables. A “classification tree” is a decision tree that places categorical variables into classes.

A “clustering algorithm” finds groups of items that are similar. For example, clustering could be used to group physiological or biochemical markers according to statistical parameters of their predictive powers for certain biological consequences. It divides a data set so that records with similar content are in the same group, and groups are as different as possible from each other. When the categories are unspecified, this is sometimes referred to as unsupervised clustering. When the categories are specified a priori, this is sometimes referred to as supervised clustering.

The term “confidence” refers to a measure of how much more likely it is that B occurs when A has occurred. It is expressed as a percentage, with 100% meaning B always occurs if A has occurred. This can also be referred to this as the conditional probability of B given A. When used with association rules, the term confidence is observational rather than predictive.

The term “gradient descent” refers to a method to find the minimum of a function of many variables.

The term “item sets” refers to a set of items that occur together.

The term “machine learning” refers to a computer algorithm used to extract usefull information from a database by building probabilistic models in an automated way.

A “model” can be descriptive or predictive. A “descriptive model” helps in understanding underlying processes or behavior. For example, an association model describes the effects of a drug on animal physiology as manifest in the measured behavior, physiology and/or biochemical markers. A “predictive model” is an equation or set of rules that makes it possible to predict an unseen or unmeasured value (the dependent variable or output) from other, known values (independent variables or input). For example, a predictive model can be used to predict side effects of a drug in humans based on data for the drug when used in non-human animals.

The term “significance” refers to a probability measure of how strongly the data support a certain result (usually of a statistical test). If the significance of a result is said to be 0.05, it means that there is only a 0.05 probability that the result could have happened by chance alone. Very low significance (less than 0.05) is usually taken as evidence that the data mining model should be accepted since events with very low probability seldom occur. So if the estimate of a parameter in a model showed a significance of 0.01 that would be evidence that the parameter must be in the model.

“Supervised learning” refers to a data analysis using a well-defined (known) dependent variable. All regression and classification techniques are supervised. In contrast, “unsupervised learning” refers to the collection of techniques where groupings of the data are defined without the use of a dependent variable. The term “test data” refers to a data set independent of the training data set, used to evaluate the estimates of the model parameters (e.g., weights).

A “time series” is a series of measurements taken at consecutive points in time. Data mining methods of the present invention that handle time series can incorporate time-related operators such as moving average. “Windowing” is used when training a model with time series data. A “window” is the period of time used for each training case.

The term “time series model” refers to a model that forecasts future values of a time series based on past values. The model form and training of the model can take into consideration the correlation between values as a function of their separation in time.

The term “training data” refers to a data set independent of the test data set, used to fine-tune the estimates of the model parameters (e.g., weights).

“Preventing vision degeneration” refers to the ability to prevent degeneration of vision in patients newly diagnosed as having a degenerative disease affecting vision, or at risk of developing a new degenerative disease affecting vision, and for preventing further degeneration of vision in patients who are already suffering from or have symptoms of a degenerative disease affecting vision.

“Promoting vision regeneration” refers to maintaining, improving, stimulating or accelerating recovery of, or revitalizing one or more components of the visual system in a manner which improves or enhances vision, either in the presence or absence of any ophthalmologic disorder, disease, or injury.

“Macular degeneration” is characterized by the excessive buildup of fibrous deposits in the macula and retina and the atrophy of the retinal pigment epithelium.

“Ophthalmic disorder” refers to physiologic abnormalities of the eye. They may involve the retina, the vitreous humor, lens, cornea, sclera or other portions of the eye, or physiologic abnormalities which adversely affect the eye, such as inadequate tear production.

“Treating a mammal for ocular neovascularization” is herein defined as treating ocular neovascularization which has already become detectable. “Mammals” are defined as humans and mammalian farm and sport animals and pets.

The terms “steroidal anti-inflammatory agent” and “glucocorticoid” are used interchangeably herein, and are meant to include steroidal agents, compounds or drugs which reduce inflammation when administered at a therapeutically effective level.

The term “inflammation-mediated condition of the eye” is meant to include any condition of the eye which may benefit from treatment with an anti-inflammatory agent, and is meant to include, but is not limited to, uveitis, macular edema, acute macular degeneration, retinal detachment, ocular tumors, fungal or viral infections, multifocal choroiditis, diabetic uveitis, proliferative vitreoretinopathy (PVR), sympathetic ophthalmia, Vogt Koyanagi-Harada (VKH) syndrome, histoplasmosis, and uveal diffusion.

The term “biodegradable polymer” refers to polymers which degrade in vivo, and wherein erosion of the polymer over time is required to achieve the agent release kinetics according to the invention. Specifically, hydrogels such as methylcellulose which act to release drug through polymer swelling are specifically excluded from the term “biodegradable polymer”.

The term “therapeutic levels” as used herein with respect to treating an ocular disorder, refers to the level of agent needed to reduce or prevent ocular injury or damage.

III. Measurement of Macular Edema

Direct Measurement of a Diseased Eye

The degree of severity of macular edema can be directly measured using state-of-the-art instruments such as confocal infrared scanning laser tomography (SLT) or optical coherence tomography (OCT).

Confocal Infrared Scanning Laser Tomography (SLT)

Confocal scanning laser tomography is a useful non-invasive diagnostic technique to quantitatively analyze macular disorders. It is especially useful for the primary assessment and follow-up studies of macular holes and central serous retinopathy.

SLT makes a quantitative measurement of a structure, such as the optic nerve, that can be viewed and assessed clinically without expensive equipment. This technology, in the form of the Heidelberg retina tomograph (HRT, Heidelberg Engineering GmbH), has been available for around 10 years. A compact version (the HRT II) has been released more recently for clinical use. The field of view is 15° and imaging can be performed through an undilated pupil. Images are monochromatic and the confocal optics enable the determination of a surface height map (topography). The margin of the optic disc is outlined by an observer and a reference plane is positioned parallel to the surface and set below the surface (Burk et al., Graefes Arch Clin Exp Ophthalmol. 238: 375-384, 2000). Structures that lie within the disc margin (contour) and above the reference plane are denoted as neuroretinal rim. Space below the reference plane is denoted as optic cup.

Scanning Laser Polarimetry

This first prototype of this instrument was developed about 10 years ago, and was first released commercially as the GDx Nerve fiber analyzer (Laser Diagnostic Technologies Inc). The second generation product is called the GDx Access. The field of view is 15° and imaging should be performed through an undilated pupil. The polarized laser scans the fundus, building a monochromatic image. The state of polarization of the light is changed (retardation) as it passes through birefringent tissue (cornea and RNFL). Corneal birefringence is eliminated (in part) by a proprietary ‘corneal compensator’. The amount of retardation of light reflected from the fundus is converted to RFNL thickness. Sub-optimal compensation of corneal birefringence is currently being addressed by the manufacturer with hardware and software modifications.

Optical Coherence Tomography (OCT) or Low-Coherence Interferometry

Optical Coherence Tomography, or OCT, is a noncontact, noninvasive imaging technique used to obtain high resolution cross-sectional images of the retina.

OCT is analogous to ultrasound B-scan imaging except that light rather than sound waves are used in order to obtain a much higher longitudinal resolution of approximately 10 μm in the retina. Imaging is performed through a dilated pupil. OCT has been shown to be clinically useful for imaging selected macular diseases including macular holes, macular edema, age-related macular degeneration, central serous chorioretinopathy, epiretinal membranes, schisis cavities associated with optic disc pits, and retinal inflammatory diseases. In addition, OCT has the capability of measuring the retinal nerve fiber layer (RNFL) thickness in glaucoma and other diseases of the optic nerve.

The first commercial application of this technology was released by Humphrey Instruments (now Zeiss Humphrey Systems) in 1995, as the Optical coherence tomography scanner. Second and third generations have been produced, giving faster scanning and greater depth resolution. The OCT 3 performs a linear scan on the retina with a near infrared (low coherence) light beam. The depth resolution is ˜10 μm. OCT software locates borders (changes in reflectivity) such as the vitreoretinal interface, the interface between RNFL and inner retinal layers, and the outer retina/choroid interface.

Diabetic Macular Edema. The topographic mapping protocol is useful for longitudinally monitoring patients for the development of macular edema and for following the resolution of edema after treatment. The false-color map of retinal thickness provides an intuitive and efficient method of comparing retinal thickness over several visits which could be directly compared with slit-lamp observation.

Laser Optical Cross-Sectioning

The commercial instrument utilizing this principle is the Retinal thickness analyzer (RTA, Talia Technology Ltd). The RTA projects a narrow slit of green laser light at an angle on the retina and acquires an image from a different angle on a digital camera. An optical cross-section of the retina is seen, with reflectance peaks that correspond to the RNFL/inner limiting membrane and the retinal pigment epithelium. The software measures the distance between the peaks to obtain retinal thickness. The macula, peripapillary area and optic disc may be scanned. Software to derive an optic disc topography has also been developed.

Comparison with a Normal Eye and Grading of Edema Severity

Fundus Photographs

Fundus photographs can be taken of the patients' eye in order to determine their macular edema assessments. The measurements can be recorded as a three digit number cmf, where c represents the thickness at the center of the macula, m represents an area of retinal thickness within 1 disc diameter of the center, and f field 2 represents the area of retinopathy within photographic. The thickness for c is recorded on a scale from 0-5. Zero shows a measurement of no thickening involving the center of the macula in the eye, 1 means that there is questionable (50-90% sign of thickening) thickening, and 2-5 are measures of definite thickness with increasing severity as the measurement approaches 5. The area of thickening in field 2 is measured on a scale ranging from 0-7. Likewise a measurement of zero means there is no thickness, 1 is questionable thickness, and 2-7 shows that there is definite thickness with increasing severity as the number increases.

Stereoscopic Photography

The only CE marked, dedicated stereoscopic optic disc camera available in the UK is the Discam (Marcher Enterprises Ltd). Stereoscopic image pairs are taken in succession at video frame rates. Newer instruments are full color and this is an advantage over all forms of scanning imaging devices (above). The field of view is 12° and pupil dilatation is required for imaging. The images provide a high magnification, stable picture that can be easier to evaluate than the image obtained with indirect ophthalmoscopy. New software enables an observer to make magnification-corrected measurements of optic disc features. The measurements are, however, subjective, and have greater between-observer variability than the semi-automated scanning devices.

Database of measurements from normal eyes are available. Such data can be used for comparison purposes.

The severity of edema can be graded based on established standards, such as the International Clinical Classification of Diabetic Retinopathy, Severity of Diabetic Macular Edema, Detailed Table (Released by International Council of Ophthalmology in October 2002, incorporated herein by reference). That scale has two major levels: Diabetic Macular Edema Absent, and Diabetic Macular Edema Present. In the latter case, it can be further divided into several levels of severity: mild, moderate, and severe Diabetic Macular Edema. The explanation of each can be found in the published standard.

IV. Delivery Means/Devices/Systems of Pharmaceutical Compositions

It should be understood that the instant invention can be used to assess the effects of a treatment on the long term outlook of visual acuity (chances of improvement vs. deterioration) in a patient with macular edema, regardless of what specific disease that treatment is intended for, and regardless of how the treatment is effected (delivered). Thus the treatment may be even directed to a condition in said patient, which condition is unrelated to said patient's eye disease, since the treatment may inherently have a side effect on said patient's eye disease. This is especially true for a drug delivered systemically. Similarly, the delivery means (local vs. systemic, sustained release vs. single or multiple administrations over a predetermined interval, etc.) also does not limit the use of the instant invention, as long as the drug may have an effect on macular edema. In addition, the specific drug used in the treatment is also non-limiting.

In one embodiment, the pharmaceutical composition is delivered through controlled release. Controlled release refers to the release of a given drug from a device at a predetermined rate. Such rate of release can be zero order, pseudo-zero order, first order, pseudo-first order and the like. Thus, relatively constant or predictably varying amounts of the drug can be delivered over a specified period of time.

Controlled delivery may take many different forms, such as intraocular injection, subretinal injection, subscleral injection, intrachoroidal injection, subconjunctival injection, etc. In controlled delivery, the delivery systems may be designed to release the pharmaceutical composition, such as glucocorticoid, at therapeutic levels to a desired location, such as the vitreous, for a sustained period of time.

Applicants have developed two proprietary platform technologies: the AEON™ and CODRUG™ systems.

The AEON™ technology, which is designed to release a drug to the affected area, could be used to deliver almost any drug that is stable at body temperature for an expected treatment period. By modifying the implant design, CDS can control both the rate and duration of release to meet a variety of different therapeutic needs. One AEON™-based product on the market to date, Vitrasert®, is FDA-approved for treating blinding CMV retinitis in AIDS patients, and has successfully demonstrated the commercial viability of the AEON™ platform. Clinical trials are underway for AEON™-based products that would treat other blinding eye diseases.

The AEON™ technology has also been adapted to develop proposed products for other debilitating conditions that are difficult to treat, including severe osteoarthritis and brain tumors.

Like the AEON™ system, the CODRUG™ platform technology allows drug release at a controlled rate over a prescribed period of time. However, the CODRUG™ technology enables the simultaneous release of drugs from the same product. Using the CODRUG™ technology, two or more drugs can be chemically linked together with a chemical bond to create novel compounds. These new compounds, when delivered to the target site, will dissolve and separate into the original drugs. A CODRUG™ library of approximately 400 drug combinations has been synthesized. Products utilizing the CODRUG™ technology could be effective in managing post-surgical pain and prostate cancer. For more details of these technologies, see U.S. Pat. Nos. 5,902,598, 6,217,895, 6,375,972, and 6,548,078, all incorporated herein by reference.

Other related patents and publications disclosing exemplary sustained release formulations and devices suitable for use, e.g., in sustained release treatment of ocular disorders, include: U.S. Pat. Nos. 6,001,386, 5,773,019, 5,681,964, 5,378,475, and PCT Publication WO02/02076.

The AEON™ platform technology gives ophthalmologists distinct options that may be less invasive, more effective and have fewer side effects for treating patients afflicted with some blinding eye diseases.

For example, CMV Retinitis is a blinding, viral eye infection that frequently occurs in AIDS patients. Vitrasert® is useful to treat CMV retinitis. The FDA-approved Vitrasert® implant (which is being marketed and sold by Bausch & Lomb) provides sustained treatment for six to eight months. It has been used in over 10,000 eyes since it was commercialized in 1996. Studies have shown that Vitrasert® is currently one of the most effective approved treatments for CMV retinitis.

Uveitis is an autoimmune condition, which manifests itself as an inflammation inside the eye, that can lead to sudden or gradual vision loss. It is estimated that more than 175,000 eyes in the U.S. suffer from severe uveitis. The FDA has granted fast-track status to the approval process for CDS's (Control Delivery Systems, Inc.) three-year Envision TD™ implant to treat this disease. Now in pivotal clinical trials, it is hoped that Envision TD™ will significantly improve visual acuity, and eliminate the need for systemic therapy, for patients with uveitis, while eliminating adverse systemic side effects.

Diabetic Macular Edema (DME) is a blinding eye disease that affects the macula, the most sensitive part of the retina, and is a major cause of vision loss in diabetics. It is estimated that over 750,000 eyes in the United States and over 1.5 million eyes outside of the United States suffer from diabetic macular edema of sufficient severity to warrant treatment. An implant designed to treat diabetic macular edema is being tested. The objectives are to reverse the disease in treated eyes, and to sustain visual acuity. The implant is now in early stage clinical trials.

ARMD (Age-Related Macular Degeneration) is the leading cause of severe visual impairment and blindness in Americans over 60, and affects more than five million people in the U.S. This condition will become increasingly prevalent as the baby-boomer generation ages. Wet ARMD, the more severe form of the disease, is responsible for approximately 85% to 90% of vision loss from ARMD.

Other controlled delivery platforms also exists. Posurdex™ is based on Oculex Pharmaceutical's proprietary biodegradable intraocular drug delivery technology. It is a biodegradable intraocular product designed to provide sustained drug therapy for an extended period of time. This unique, micro-sized product is inserted directly inside the eye by a physician to ensure that therapeutic levels of medication are delivered to the targeted site over a predetermined amount of time. For more details, see U.S. Pat Nos. 5,443,505, 5,632,984, 5,766,242, 5,824,072, 5,869,079, 6,331,313, and 6,369,116, all incorporated herein by reference.

Using these and other equivalent technologies, a number of drugs and other pharmaceutical compounds can be effectively delivered as an implant to a patient, especially in the eye. These compounds include steroidal anti-inflammatory agents, anesthetics, analgesics, cell transport/mobility impending agents such as colchicine, vincristine, cytochalasin B and related compounds; antiglaucoma drugs including beta-blockers such as timolol, betaxolol, atenolol, etc; carbonic anhydrase inhibitors such as acetazolamide, methazolamide, dichlorphenamide, diamox; and neuroprotectants such as nimodipine and related compounds.

Exemplary steroidal anti-inflammatory agents can be selected from 2,1,-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide. In a preferred embodiment, the steroidal antiinflammatory agent is selected from cortisone, dexamethasone, hydrocortisone, methylprednisolone, prednisolone, prednisone, and triamcinolone. In a more preferred embodiment, the steroidal antiinflammatory agent is dexamethasone. In another embodiment, the biodegradable implant comprises more than one steroidal anti-inflammatory agent.

The implants may further comprise one or more additional therapeutic agents, such as antimetabolites and/or antibiotics.

Antimetabolites include, but are not limited to, folic acid analogs (e.g., denopterin, edatrexate, methotrexate, piritrexim, pteropterin, Tomudex, trimetrexate), purine analogs (e.g., cladribine, fludarabine, 6-mercaptopurine, thiamiprine, thiaguanine), and pyrimidine analogs (e.g., ancitabine, azacitidine, 6-azauridine, carmofur, cytarabine, doxifluridine, emitefur, enocitabine, floxuridine, fluorouracil, genicitabine, tegafur).

Specific antibiotics include, but are not limited to:

Antibacterial Antibiotics:

Aminoglycosides (e.g., amikacin, apramycin, arbekacin, bambermycins, butirosin, dibekacin, dihydrostreptomycin, fortimicin(s), gentainicin, isepamicin, kanamycin, micronomicin, neomycin, neomycin undecylenate, netilinicin, paroinomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), P-lactams (e.g., carbacephems e.g., loracarbef), carbapenems (e.g., biapenem, imipenem, tneropenem, panipenem), cephalosporins (e.g., cefaclor, cefadroxil, cefamandole, cefatrizine, cefazedone, cefazolin, cefeapene, pivoxil, cefclidin, cefdinir, cefditoren, cefepime, cefetamet, cefixime, cefinenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefterain, ceftezole, ceftibuten, ceftizoxime, ceftriaxone, cefuroxime, cefuzonam, cephacetrile sodium, cephalexin, cephaloglycin, oephaloridine, cephalosporin, cephalothin, cephapirin sodium, cephradine, piveefalexin), cephamycins (e.g., cefbuperazone, cefinetazole, ceftninox, cefotetan, cefoxitin), monobactains (e.g., aztreonarn, carumonam, tigemonam), oxacephems, flomoxef, moxalactam), penicillins (e.g., anidinocillin, amdinocillin pivoxil, arnoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafeillin sodium, oxacillin, penamecillin, penethamate hydriodide, penicillin g benethamine, penicillin g benzathine, 12 penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), fincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusaftmgine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

It also includes: tetracycline, chlortetracycline, bacitracin, neomycin, polymyxin, gramicidin, oxytetracycline, chloramphenicol, gentamycin, and erythromycin; antibacterials such as sulfonamides, sulfacetamide, sulfamethizole and sulfisoxazole; anti-fungal agents such as fluconazole, nitrofurazone, amphotericine B, ketoconazole, and related compounds; anti-viral agents such as trifluorothymidine, acyclovir, ganciclovir, DDI, AZT, foscamet, vidarabine, trifluorouridine, idoxuridine, ribavirin, protease inhibitors and anti-cytomegalovirus agents; antiallergenics such as methapyriline; chlorpheniramine, pyrilamine and prophenpyridamine; anti-inflammatories such as hydrocortisone, dexamethasone, fluocinolone, prednisone, prednisolone, methylprednisolone, fluorometholone, betamethasone and triamcinolone; decongestants such as phenylephrine, naphazoline, and tetrahydrazoline; miotics and anti-cholinesterases such as pilocarpine, carbachol, di-isopropyl fluorophosphate, phospholine iodine, and demecarium bromide; mydriatics such as atropine sulfate, cyclopentolate, homatropine, scopolamine, tropicamide, eucatropine; sympathomimetics such as epinephrine and vasoconstrictors and vasodilators. Anticlotting agents such as heparin, antifibrinogen, fibrinolysin, anti clotting activase, etc., can also be delivered.

Synthetic Antibacterials.

2,4-Diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, niftirtoinol, nitrofurantoin), quinolones and analogs (e.g., cinoxacin, ciprofloxacin, clinafloxacin, difloxacin, enoxacin, fieroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacm, tosufloxacin, trovafloxacin), sulfonamides (e.g., acetyl sulfamethoxypyrazine, benzylsulfamide, chloramine-b, chloramine-t, dichloramine t, n 2formylsulfisomidine, n4-p-d-glucosylsulfanilamide, mafenide, 4′(methylsulfamoyl)sulfanilanilide, noprylsulfamide, phthalylsulfacetamide, 13 phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfainidochrysoidine, sulfamoxole, sulfanilamide, 4sulfanilamidosalicylic acid, n4-sulfanilylsulfanilamide, sulfanilylurea, nsulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene-citrate, methenamine hippurate, methenamine mandelate, methenamine sulfosalicylate, nitroxoline, taurolidine, xibomol).

Antifungal Antibiotics.

Polyenes (e.g., amphotericin b, candicidin, dermostatin, filipin, fimgichromin, hachimycin, hamycin, lueensomycin, mepartricin, natamycin, nystatin, pecilocin, perimycin), others (e.g., azaserine, griseofulvin, oligomycins, neomycin undecylenate, pyrrolnitrin, siccanin, tubercidin, viridin).

Synthetic Antifungals.

Allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole) others (e.g., acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, 14 loflucarban, nifuratel, potassium iodide, propionic acid, pyrithione, salicylanilide, sodium propionate, sulbentine, tenonitrozole, triacetin, ujothion, undecylenic acid, zinc propionate).

Antineoplastic.

Antibiotics and analogs (e.g., aclacinomycins, actinomycin fl, anthramycin, azaserine, bleomycins, cactinomycin, carubicin, carzinophilin, chromomycins, dactinomycin, daunorubicin, 6-diazo OXO-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorabicin), antimetabolites (e.g., folic acid analogs (e.g., denopterin, edatrexate, methotrexate, piritrexim, pteropterin, Tomudex, trimetrexate), purine analogs (e.g., cladribine, fludarabine, 6-mercaptopurine, thiamiprine, thioguanine), pyrimidine analogs (e.g., ancitabine, azacitidine, 6-azauridine, carmofur, cytarabine, doxifluridine, emitefur, enocitabine, floxuridine, fluorouracil, genicitabine, tagafur).

Additional antidiabetic agents that may be delivered using the present devices include acetohexamide, chlorpropamide, glipizide, glyburide, tolazamide, tolbutamide, insulin, aldose reductase inhibitors, etc. Some examples of anti-cancer agents include 5-fluorouracil, adriamycin, asparaginase, azacitidine, azathioprine, bleomycin, busulfan, carboplatin, carmustine, chlorambucil, cisplatin, cyclophosphamide, cyclosporine, cytarabine, dacarbazine, dactinomycin, daunorubicin, doxorubicin, estramustine, etoposide, etretinate, filgrastin, floxuridine, fludarabine, fluorouracil, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostin, streptozocin, tamoxifen, taxol, teniposide, thioguanine, uracil mustard, vinblastine, vincristine and vindesine.

Hormones, peptides, nucleic acids, saccharides, lipids, glycolipids, glycoproteins, and other macromolecules can be delivered using the present devices. Examples include: endocrine hormones such as pituitary, insulin, insulin-related growth factor, thyroid, growth hormones; heat shock proteins; immunological response modifiers such as muramyl dipeptide, cyclosporins, interferons (including alpha, beta, and gamma interferons), interleukin-2, cytokines, FK506 (an epoxy-pyrido-oxaazacyclotricosine-tetrone, also known as Tacrolimus), tumor necrosis factor, pentostatin, thymopentin, transforming factor beta₂, erythropoetin; antineogenesis proteins (e.g., VEGF, Interferons), among others and anticlotting agents including anticlotting activase. Further examples of macromolecules that can be delivered include monoclonal antibodies, brain nerve growth factor (BNGF), ciliary nerve growth factor (CNGF), vascular endothelial growth factor (VEGF), and monoclonal antibodies directed against such growth factors. Additional examples of immunomodulators include tumor necrosis factor inhibitors such as thalidomide.

Examples of corticosteroids useful in the present invention include, for example, triamcinolone, dexamethasone, fluocinolone, cortisone, prednisolone, flumetholone, and derivatives thereof. Preferred corticosteroids include triamcinolone acetonide (TA) or fluocinolone acetonide (FA).

In addition, nucleic acids can also be delivered wherein the nucleic acid may be expressed to produce a protein that may have a variety of pharmacological, physiological or immunological activities. Thus, the above list of drugs is not meant to be exhaustive. Practically any drug may be used in the instant invention, and there are no particular restrictions in terms of molecular weight and so forth.

V. Use in Different Types of Diseases

A wide variety of systemic and ocular conditions such as inflammation, infection, cancerous growth, may be prevented or treated using the drug delivery devices described above. More specifically, ocular conditions such as glaucoma, proliferative vitreoretinopathy, diabetic retinopathy, uveitis, keratitis, cytomegalovirus retinitis, herpes simplex viral and adenoviral infections can be treated or prevented.

It should be understood that the present invention can be used in assessing treatment for a number of ophthalmic diseases, such as disorders of the retina, including but not limited to: vascular retinopathies (e.g., arteriosclerotic retinopathy and hypertensive retinopathy), central and branch retinal artery occlusion, central and branch retinal vein occlusion, diabetic retinopathy (e.g., proliferative retinopathy and not proliferative retinopathy), macular degeneration of the aged (age-related macular degeneration or senile macular degeneration), neovascular macular degeneration, retinal detachment, retinitis pigmentosa, retinal photic injury, retinal ischemia-induced eye injury, and glaucoma (e.g., primary glaucoma, chronic open-angle glaucoma, acute or chronic angle-closure, congenital (infantile) glaucoma, secondary glaucoma, and absolute glaucoma); disorders of the uveal tract, such as uveitis (inflammation of the uveal tract or retina), anterior uveitis, intermediate uveitis, posterior uveitis, iritis, Cyclitis, choroiditis, ankylosing spondylitis, Reiter's syndrome, pars planitis, toxoplasmosis, cytomegalovirus (CMV), acute retinal necrosis, toxocariasis, birdshot choroidopathy, histoplasmosis (presumed ocular histoplasmosis syndrome), Behcet's syndrome, sympathetic ophthalmia, VogtKoyanagi-Harada syndrome, sarcoidosis, reticulum cell sarcoma, large cell lymphoma, syphilis, tuberculosis, juvenile rheumatoid arthritis, endophthalmitis, and malignant melanoma of the choroid.

In a preferred embodiment, the instant invention is used to assess the treatment for posterior uveitis, Diabetic Macular Edema (DME), Wet ARMD, or CMV retinitis.

Preferred inflammation-mediated conditions of the eye which may be treated by the methods described above include uveitis, macular edema, acute macular degeneration, retinal detachment, ocular tumors, flitigal or viral infections, multifocal choroiditis, diabetic uveitis, proliferative vitreoretinopathy (PVR), sympathetic ophthalmia, Vogt Koyanagi-Harada (VKH) syndrome, histoplasmosis, and uveal diffusion. In a preferred embodiment, the inflammation-mediated condition of the eye is uveitis. In another preferred embodiment, the inflammation-mediated condition of the eye is proliferative vitrioretinopathy (PVR).

The method described above is particularly effective in treating diseases of the retina, retinal pigment epithelium (RPE) and choroid. These diseases include, for example, ocular neovascularization, ocular inflammation and retinal degenerations. Specific examples of these disease states include diabetic retinopathy, chronic glaucoma, retinal detachment, sickle cell retinopathy, senile macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases retinal ischemia, choroidal vascular insufficiency, choroidal thrombosis, neovascularization of the optic nerve, diabetic macular edema, cystoid macular edema, macular edema, retinitis pigmentosa, retinal vein occlusion, proliferative vitreoretinopathy, angioid streak, and retinal artery occlusion, and, neovascularization due to penetration of the eye or ocular injury.

Some of the representative (but non-limiting) diseases are described in more detail below.

Macular Degeneration

Age-related macular degeneration (AMD) is the major cause of severe visual loss in United States citizens over the age of 55. Most AMD patients have a build up of deposits within and under the retinal pigment epithelium in the macular region resulting in atrophy of the retina and the retinal pigment epithelium. The retinal pigment cells are long-lived. They scavenge for photoreceptor discs from the rods and cones for years and accumulate intracellular wastes. The incompletely digested residues reduce cytoplasmic space (Feeny-Bums, L. et al., Invest. Ophthal. Mol. Vis. Sci. (1984) 25:195-200) and interfere with metabolism. As the cell volume available to the organelles diminishes, the capacity to digest photoreceptors decreases, and this may be the basis for macular degeneration.

Some patients also experience exudative AMD with choroidal neovascularization, detachment and tears of the retinal pigment epithelium, fibrovascular scarring, and vitreous hemorrhage. This process is responsible for more than 80% of cases of significant visual loss in patients with AMD.

Age-related macular degeneration (AMD) is a sight-threatening disorder which occurs in either an atrophic or (less commonly) an exudative form. In exudative AMD, blood vessels grow from the choriocapillaris through defects in Bruch's membrane, and in some cases the underlying retinal pigment epithelium (RPE). Organization of serous or hemorrhagic exudates escaping from these vessels results in fibrous scarring of the macular region with attendant degeneration of the neuroretina and permanent loss of central vision. Wet ARMD, the more severe form of the disease, is responsible for approximately 85% to 90% of vision loss from ARMD.

Other Retinal Disorders

Other retinal disorders include edema and ischemic conditions. Macular and retinal edema are often associated with metabolic illnesses such as diabetes mellitus. Retinal edema is found in a large percentage of individuals who have undergone cataract extraction and other surgical procedures upon the eye. Edema is also found with accelerated or malignant hypertension. Macular edema is a common complication of prolonged inflammation via uveitis, Eales disease, or other diseases. Local edema is associated with multiple cytoid bodies (“cotton bodies”) as a result of AIDS.

Retinal ischemia can occur from either choroidal or retinal vascular diseases, such as central or branch retinal vein occlusion, collagen vascular diseases and thrombocytopenic purpura. Retinal vasculitis and occlusion is seen with Eales disease and systemic lupus erythematosus.

Proliferative Diabetic Retinopathy (PDR)

Sebag and McMeel reviewed the pathogenesis of PDR (Survey of Ophthalmol. (1986) 30:377-84). The initiating event may be inadequate tissue oxygenation which causes vasodilation. Inadequate oxygenation may occur after the arterial basement membrane has thickened with diabetes-related deposits and because of endothelial cell proliferation, which is associated with pericyte degeneration. Basement membrane thickening and loss of pericytes are believed to result from low insulin and hyperglycemia, two important metabolic abnormalities of diabetes.

The neovascularization of PDR has been attributed to the subtle vascular abnormalities described above. Even this slight disruption may permit normally absent chemicals to enter the eye across the blood-retinal barrier.

Several growth factors besides TGF-beta appear to be involved in diabetic retinopathy, including fibroblast growth factors (FGF), an interplay of FGF and TGF-beta, tumor necrosis factor (TNF-alpha and beta), which are known to have angiogenic properties. (Wiedemann, Survey of Ophthalmol. (1992) 36:373-84). Others have proposed that because retinal blood vessels appear to have a unique response to diabetic ischemia, there may be specific retina-derived growth factors. Berritault et al. Differentiation (1981) 18:29-42; Chen and Chen Invest. Ophthalmol. Vis. Sci. (1980) 19: 596-02; D's Amore and Klagsbum J. Cell. Biol. (1984) 99: 1545-49; Elstow et al. Invest. Ophthalmol. Vis. Sci. (1985) 26:74-79; Glaser et al. Ophthalmology (1980) 87:440-46; and Ruelius-Altemose et al. Invest. Ophthalmol. Vis. Sci. (1985) 26 (ARVO Suppl):25.

Potential inhibitors of retinal angiogenesis have been sought. Tumor-induced angiogenesis was prevented with an extract of cartilage, which weighed about 16,000 daltons and inhibited protease activity. Langer et al. Science (1976) 193:70-71. Later studies indicated that normal vitreous humor contained such an inhibitor. For example, a vitreous protein with a molecular weight of 6200 was found to inhibit RDGF-induced proliferation and thymidine incorporation by vascular endothelial cells in vitro. Raymond and Jacobson, Exp. Eve Res. (1982) 34:267-86.

Clinically, the appearance of cotton wool spots in the retina signifies the onset of retinal ischemia. Sebag and McMeel, ibid. These spots are irregular patches of fibrous tissue.

Uveitis

Uveitis refers to inflammation of the uveal tract. It includes iritis, cyclitis and iridocyclitis and choroiditis and usually occurs with inflammation of additional structures of the eye. This disorder has a variety of causes but is typically treated with systemic steroids, topical steroids or cyclosporin. The disease frequently presents with a chronic inflammation occurring either in the anterior segment (70%) or in the posterior segment (30%) which is complicated by episodes of severe exacerbation that may not be controllable with conventional medications. Reports in the literature suggest that 30,000 individuals become legally blind each year in the United States from uveitis. In addition, an estimated 20,000 individuals suffer significant loss of visual acuity from this disorder. Additional means to control this condition, without suppressing infection fighting abilities with steroids, would be highly beneficial.

VI. Data Generation and Analysis

A. Patient Data

The patient data can include data pertaining to behavioral, neurological, biochemical and/or physiological activity or markers. For instance, the data can include one or more of sleeping, locomotion (including ambulatory and non-ambulatory movements, foot misplacement, and the like), body weight, anxiety, pain sensitivity, convulsions, cardiac response (e.g., output, QT interval), heart rate, blood pressure and body temperature, respiration (e.g., rate, O₂ or CO₂), circadian rhythms, learning, memory (short term/long term) and the like.

The subject methods can also utilize cellular and molecular marker data., such as for example, changes in gene expression, levels of proteins, post-translational modification of proteins or other cellular structures (including extracellular markers), extracellular matrix composition or levels, tissue microarchitecture, metabolites, hormones or other natural small molecules. Rates of cell growth, differentiation and/or death may be useful in identifying certain surrogate endpoints.

By measuring a plurality of responses the methods of this invention provide a means for objectively finding surrogate markers which are predictive of changes a drug may induce in a patient.

B. Database Analysis Techniques

Various data mining techniques can be used as part of the subject invention. In certain preferred embodiments, the data mining system uses classification techniques, such as clustering algorithms, which find rules that partition the database into finite, disjoint, and previously known (or unknown) classes. In other embodiments, the data mining system uses association techniques, e.g., of summarization algorithms, which find the set of most commonly occurring groupings of items. Yet in other embodiments, the data mining system uses overlapping classes.

In one embodiment, the subject method uses a data mining technique based on association rules algorithms. These techniques derive a set of association rules of the form X→Y, where X and Y are sets of behavioral, neurological, biochemical and/or physiological responses and each drug administration is a set of literals. The data mining task for association rules can be broken into two steps. The first step consists of finding all large item-sets. The second step consists of forming implication rules with a user specified confidence among the large item-sets found in the first step. For example, from a dataset, one may find that an association rule such as drugs which slowed a decrease in visual acuity also cause a reduction in the rate of retinal thickening, or a decrease in intraocular pressure. Association rules can also be more complex, requiring that two or more criteria are met in order for the rule to be evoked. A rule X →Y holds in the data set D with confidence c if c % of the occurrences of X in the data set also contain Y. The rule X→Y has support s in the data set if s % of the entries in D contain X→Y. Confidence is a measure of the strength of implication and support indicates the frequencies of occurring patterns in the rule.

Another technique that can be used in the methods of the present invention is the process of data classification. Classification is the process of finding common properties among a set of “objects” in a database, and grouping them into various classes based on a classification scheme. Classification models are first trained on a training data set which is representative of the real data set. The training data is used to evolve classification rules for each class such that they best capture the features and traits of each class. Rules evolved on the training data are applied to the main database and data is partitioned into classes based on the rules. Classification rules can be modified as new data is added.

Yet another data mining technique that can be used in the subject method is the use of sequential pattern mining. This technique can be used to find sequential patterns which occur a significant number of times in the database. This analysis can be used to detect temporal patterns, such as the manifestation of secondary adaptation or effects involving combinatorial therapies. Time-Series clustering is another data mining technique that can be used to detect similarities in different time series.

In yet another embodiment, the subject method uses a clustering method for finding correlations in the behavioral database(s). In general, clustering methods can be broadly classified into partitional and hierarchical methods.

Partitional clustering attempts to determine k partitions that optimize a certain criterion finction. The square-error criterion is a good measure of the within-cluster variation across all the partitions. The objective is to find k partitions that minimize the square-error. Thus, square-error clustering tries to make the k clusters as compact and separated as possible, and works well when clusters are compact clouds that are rather well separated from one another.

Hierarchical clustering is a sequence of partitions in which each partition is nested into the next partition in the sequence. An agglomerative method for hierarchical clustering starts with the disjoint set of clusters, which places each input data point in an individual cluster. Pairs of clusters are then successively merged until the number of clusters reduces to k. At each step, the pair of clusters merged are the ones between which the distance is the minimum. There are several measures used to determine distances between clusters. For example, pairs of clusters whose centroids or means are the closest are merged in a method using the mean as the distance measure (dₘₑₐₙ). This method is referred to as the centroid approach. In a method utilizing the minimum distance as the distance measure, the pair of clusters that are merged are the ones containing the closest pair of points (dₘᵢₙ). This method is referred to as the all-points approach.

In another embodiment, the subject method uses Principal Component Analysis (PCA). This is not a classification method per se. The purpose of PCA is to represent the variation in a data set into a more manageable form by recognizing classes or groups. The assumption in PCA is that the input is very high dimensional (tens or even thousands of variables). PCA extracts a smaller number of variables that cover most of the variability in the input variables. As an example, suppose there are data along a line in 3-space. Normally one would use 3 variables to specify the coordinates of each data point. In fact, just 1 variable is needed: the position of the data point along the line that all the data lies on. PCA is a method for finding these reductions. An advantage to PCA is that it can be a reasonably efficient method whose reduction is well founded in terms of maximizing the amount of data variability explained with use of a smaller number of variables.

Still another embodiment utilizes a neural net or neural network, e.g., a complex non-linear function with many parameters that maps inputs to outputs. Such algorithms may use gradient descent on the number of classification errors made, e.g., a routine is implemented such that the number of errors made decreases monotonically with the number of iterations. Gradient descent is used to adjust the parameters such that they classify better. An advantage to neural nets is that such algorithms can handle high dimensional, non-linear, noisy data well.

The neural net can be trained with “supervision”, e.g., a mechanism by which the net is given feedback by classifying its responses as “correct” or “incorrect”. It eventually homes into the correct output for each given input, at least with some probability. Such machine learning techniques may be advantageously employed for either or both of vision classification components or data mining components of the instant invention.

Supervised learning requires the buildup of a library of readily classified data sets for input into the neural net. Although more economic in terms of the amount of data needed, supervised learning implies that only pre-determined classes can be ascribed to unseen data. To allow for the possibility of finding a novel therapeutic class, such as “antidepressant drugs with anti-manic component” unsupervised clustering could be more appropriate.

In certain embodiments, a preferred method can combine both types of learning: a supervised learning of the neural net until it correctly classifies a basic training set but which also utilizes unsupervised learning to further subdivide the trained classes into meaningful sub-classes, or to add completely new sub-classes.

Principal component analysis (PCA) involves a mathematical procedure that transforms a number of (possibly) correlated variables into a (smaller) number of uncorrelated variables called principal components. The first principal component accounts for as much of the variability in the data as possible, and each successive component accounts for as much of the remaining variability as possible. Traditionally, principal component analysis is performed on a square symmetric matrix of type SSCP (pure sums of squares and cross products), Covariance (scaled sums of squares and cross products), or Correlation (sums of squares and cross products from standardized data). The analysis results for matrices of type SSCP and Covariance do not differ. A Correlation object is preferably used if the variances of individual variates differ much, or the units of measurement of the individual datapoints differ, such as is the case when the analysis comprises data from behavioral, neurological, biochemical and physiological measures. The result of a principal component analysis on such objects will be a new object of type PCA.

In still other embodiments, the subject method utilizes K-means and fuzzy clustering. Gaussian mixture models are a common version of this. These techniques are “unsupervised” clustering methods. They assume the user has no outputs, but would like to group the data anyway according to inputs that are similar to each other. The idea is to choose a model for each cluster. For example, each cluster may consist of points inside a hyper-sphere centered at some location in the input space. These methods automatically determine the number of clusters, place them in the correct places, and determine which points belong to which clusters. An advantage to these techniques is that they can be efficient algorithms and can do a good job of finding clusters. This is a method of choice when the user does not have a priori information about the classes

Another embodiment utilizes the hierarchical clustering Serial Linkage Method. This is an unsupervised clustering method in the same sense as K-means and fuzzy clustering. Here individual points are joined to each other by being close to each other in the input space. As these points are joined together, they define clusters. As the algorithm continues, the clusters are joined together to form larger clusters. Compared to K-means and fuzzy clustering, hierarchical clustering has the advantage that clusters can have arbitrary non-predefined shapes and the result correctly shows “clusters of clusters.” A disadvantage to these methods is they tend to be more sensitive to noise.

Yet another embodiment utilizes a nearest neighbor algorithm. This is a true supervised learning method. There is a set of training data (inputs, e.g., datapoints, and outputs, e.g., classes) that are given in advance and just stored. When a new query arrives, the training data is searched to find the single data point whose inputs are nearest to the query inputs. Then the output for that training data point is reported as the predicted output for the query. To reduce sensitivity to noise, it is common to use “k” nearest neighbors and take a vote from all their outputs in order to make the prediction.

In yet another embodiment, the subject method uses a logistic regression algorithm. This is related to linear regression (fitting a line to data), except that the output is a class rather than a continuous variable. An advantage is that this method provides a statistically principled approach that handles noise well.

Still another embodiment utilizes a Support Vector Machine algorithm. This also has a linear separator between classes, but explicitly searches for the linear separator that creates the most space between the classes. Such techniques work well in high dimensions. Yet another embodiment relies on a Bayes Classifier algorithm. The simplest form is a naive Bayes classifier. These algorithms build a probabilistic model of the data from each class. Unsupervised methods above may be used to do so. Then, based on a query, the model for each class is used to calculate the probability that that class would generate the query data. Based on those responses, the most likely class is chosen.

Yet another embodiment utilizes a Kohonen self organizing maps (SOM) Clustering algorithm. These algorithms are related to neural nets in the sense that gradient descent is used to tune a large number of parameters. The advantages and disadvantages are similar to those of neural networks. In relation to neural networks, Kohonen SOM clustering algorithms can have the advantage that parameters can be more easily interpreted, though such algorithms may not scale up to high dimensions as well as neural nets can.

The subject databases can include extrinsically obtained data, such as known protein interactions of a drug, chemical structure, K_{d} values, Pₖ/P_{d} parameters, IC₅₀ values, ED₅₀ values, TD₅₀ values and the like.

The system of the present invention can also provide tools for visualizing trends in the dataset, e.g., for orienteering, to simplify user interface and recognition of significant correlations.

### EXAMPLE

Analysis of 12-Month Data from Clinical Trial of Fluocinolone Acetonide Implant (Retisert™) in the Treatment of Diabetic Macular Edema (DME)

Presented below are results of the intent-to-treat analysis of 12-month data for the first phase III randomized, controlled and masked clinical trial designed to assess the safety and efficacy of the Retisert™ implant for the treatment of diabetic macular edema (DME).

In this multi-center trial, 80 patients were randomized to receive standard of care (macular grid laser or observation) or either a 0.5 mg or a 2 mg Retisert™ implant. The implant is a tiny drug reservoir implanted into the back of the eye that delivers sustained and consistent levels of the drug fluocinolone acetonide directly to the affected area of the eye for up to three years. Enrollment of patients for the 2 mg dose was discontinued early in the DME trial.

Key elements of the clinical trial data are:

Primary Endpoint: Macular Edema/Retinal Thickening

Edema is caused by a build-up of fluid in the retina that can affect the photoreceptor nerve cells lining the back of the eye, resulting in impaired vision. This study was appropriately designed and powered to demonstrate a difference in the resolution of edema (as evidenced by a score of zero for retinal thickness at the center of the macula) between patients treated with the Retisert™ implant and those treated with the standard of care. At the 12-month follow-up, 48.8% of the patients treated with the 0.5 mg implant had a reduction of their retinal thickness scores to zero (resolution of macular edema), compared to 25.0% of those receiving standard of care (p<0.05). This is an almost 100% improvement over the standard of care.

Secondary Endpoints

Although not designed or powered to demonstrate improvement in visual acuity and other secondary endpoints, these measures were evaluated and differences assessed between patients treated with the 0.5 mg implant and those treated with standard of care.

At 12 months, patients treated with the 0.5 mg implant were more likely to show improvement in visual acuity of 15 letters or more compared to patients treated with the standard of care (19.5% vs. 7.1%). Although this result has not reached statistical significance, possibly due to the size of the sample, this apparent improvement of almost 200% increase is rather encouraging. Similarly, implant-treated patients were less likely to have a decrease of 15 or more letters of visual acuity than were those in the standard of care group: 4.9% versus 14.3%. Again, although the data did not reach statistical significance, possibly due to sample size limitation, this decrease of 66% is also very encouraging. Over 70% of patients treated with the 0.5 mg implant had improved or stable visual acuity compared to 50% treated with standard of care (p=0.08).

More patients in the standard of care group had a worsening of their diabetic retinopathy score at twelve months (29.6%) compared to those receiving the 0.5 mg implant (5.1%).

These unexpected data indicates that there is a correlation between the reduction of short term macular edema with an increased long term benefit of improvement in visual acuity, and/or a decreased long term risk of deterioration in visual acuity.

Adverse Events

The overall incidence of serious ocular adverse events in the study eye over 12 months was 58.5% in patients receiving the 0.5 mg implant and 10.7% in the standard of care group. These events, which were anticipated for implant patients given the nature of the disease and the type of drug used, included increase in intraocular pressure (IOP), vitreous hemorrhage and cataracts. The proportion of patients with increased intraocular pressure in the study eye was higher in the 0.5 mg group (19.5%) than in the standard of care group (0.0%). Five of eight patients with elevated IOP requiring treatment were successfully managed with anti-hypertensive medication; three patients required trabeculectomy. In addition, cataract progression at 12 months was 0.0% in the standard of care group vs. 54.8% of the 31 patients in the 0.5 mg implant group who had not previously undergone cataract surgery. No patients required implant removal or withdrew from the study due to an adverse event.

Patients in this trial will be followed for an additional three years to continue to monitor the safety of the implant over an extended period of time.

Incorporation By Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents Those skilled in the art will recognize, or be able to ascertain using no more than routine experimentation, many equivalents to the specific embodiments of the invention described herein. Such equivalents are intended to be encompassed by the following claims.

We claim:

1. A packaged pharmaceutical comprising:
(A) a pharmaceutical formulation including one or more drugs that may affect visual acuity;
(B) instructions for assessing a patient to whom said pharmaceutical formulation is administered and who presents some degree of macular edema, said instructions providing for altering dosage regimen and/or discontinuing administration if the degree of macular edema does not decrease after administration of said formulation, changes in said degree of macular edema being predictive for long term changes in visual acuity.

2. The packaged pharmaceutical of claim 1, wherein said pharmaceutical formulation is a sustained-release formulation.

3. The packaged pharmaceutical of claim 2, wherein said pharmaceutical formulation is provided in a sustained-release device.

4. The packaged pharmaceutical of claim 1, wherein said pharmaceutical formulation is for treating an ophthalmic disorder.

5. The packaged pharmaceutical of claim 4, wherein said ophthalmic disorder is:
posterior uveitis, Diabetic Macular Edema (DME), Wet ARMD, or CMV retinitis.

6. The packaged pharmaceutical of claim 4, wherein said pharmaceutical formulation is for intraocular injection or implantation.

7. The packaged pharmaceutical of claim 1, wherein said pharmaceutical formulation comprises one or more of an anti-inflammatory compound, neuroprotective agent, and/or immunomodulatory compounds.

8. The packaged pharmaceutical of claim 1, wherein said pharmaceutical formulation includes a corticosteroid.

9. The packaged pharmaceutical of claim 8, wherein said corticosteroid is:
triamcinolone, dexamethasone, fluocinolone, cortisone, prednisolone, flumetholone, or derivatives thereof.

10. The packaged pharmaceutical of claim 8, wherein said corticosteroid is triamcinolone acetonide (TA) or fluocinolone acetonide (FA).

11. The packaged pharmaceutical of claim 1, wherein said instructions for assessing the patient include instructions to measure the area, volume, thickness (height or elevation) of the macular edema.

12. The packaged pharmaceutical of claim 1, wherein said instructions set forth clearance of edema as being predictive of lower percentage of patients with greater than or equal to a 15 letter loss in visual acuity.

13. The packaged pharmaceutical of claim 2, wherein said sustained-release formulation is capable of being released over a period of about 1 month to about 20 years, preferably over a period of about 6 months to about 5 years.

14. The packaged pharmaceutical of claim 1, wherein said instructions include monitoring the degree of macular edema in said patient for about 2-18 months, preferably 6-12 months.

15. The packaged pharmaceutical of claim 3, wherein the sustained release device is a biocompatible implantable ocular controlled release drug delivery device sized for implantation within an eye for continuously delivering said pharmaceutical formulation within the eye for a period of at least several weeks, which device comprises a polymeric outer layer that is substantially impermeable to the drug and ocular fluids covering a core comprising pharmaceutical formulation, wherein said outer layer has one or more orifices that create a flow path through which fluids may pass to contact the core and dissolved drug may pass to the exterior of the device.

16. The packaged pharmaceutical of claim 15, wherein the device further includes one or more semi-permeable layers disposed in said flow path, which semi-permeable layers are at least partially permeable to dissolved drug, wherein said semi-permeable layers reduce influx of proteins from ocular fluid and/or reduce the rate of release of dissolved drug from the device.

17. The packaged pharmaceutical of claim 15, wherein the rate of release of drug is determined solely by the composition of the core and the total surface area of the one or more orifices relative to the total surface area of said device.

18. The packaged pharmaceutical of claim 15, wherein said outer layer comprises polytetrafluoroethylene, polyfluorinated ethylenepropylene, polylactic acid, polyglycolic acid, or silicone or a mixture thereof.

19. The packaged pharmaceutical of claim 15, wherein the outer layer is biodegradable.

20. The packaged pharmaceutical of claim 16, wherein said semipermeable layer comprises PVA.

21. The packaged pharmaceutical of claim 2, wherein the sustained release formulation is a biodegradable implant comprising said one or more drugs and a biodegradable polymer.

22. The packaged pharmaceutical of claim 21, wherein said one or more drugs comprise about 50-80 weight percent of the implant.

23. A method for assessing the long term effect on visual acuity (VA) of a pharmaceutical formulation for treatment in a patient who presents some degree of macular edema, the method comprising assessing degree of macular edema before and after said treatment, wherein a reduction in said severity is predictive of increased long term benefit of improvement in visual acuity, and/or decreased long term risk of deterioration in visual acuity.

24. The method of claim 23, wherein said pharmaceutical formulation is a sustained-released formulation.

25. The method of claim 24, wherein said pharmaceutical formulation is provided in a sustained-release device.

26. The method of claim 23, wherein said pharmaceutical formulation is for treating an ophthalmic disorder.

27. The method of claim 26, wherein said ophthalmic disorder is: posterior uveitis, Diabetic Macular Edema (DME), Wet ARMD, or CMV retinitis.

28. The method of claim 23, wherein said treatment is directed to a condition unrelated to an ophthalmic disorder, and wherein said effect is a side effect of said treatment.

29. The method of claim 26, wherein said pharmaceutical formulation is for intraocular injection or implantation.

30. The method of claim 23, wherein said pharmaceutical formulation comprises one or more of an anti-inflammatory compound, neuroprotective agent, and/or immunomodulatory compounds.

31. The method of any of claim 23, wherein said pharmaceutical formulation includes a corticosteroid.

32. The method of claim 31, wherein said corticosteroid is: triamcinolone, dexamethasone, fluocinolone, cortisone, prednisolone, flumetholone, or derivatives thereof.

33. The method of claim 31, wherein said corticosteroid is triamcinolone acetonide (TA) or fluocinolone acetonide (FA).

34. The method of claim 23, wherein said instructions said instructions for assessing the patient include instructions to measure the area, volume, thickness (height or elevation) of the macular edema.

35. The method of claim 23, wherein clearance of edema after said treatment is predictive of lower percentage of patients with greater than or equal to a 15 letter loss in visual acuity.

36. The method of claim 25, wherein the sustained release device is a biocompatible implantable ocular controlled release drug delivery device sized for implantation within an eye for continuously delivering said pharmaceutical formulation within the eye for a period of at least several weeks, which device comprises a polymeric outer layer that is substantially impermeable to the drug and ocular fluids covering a core comprising pharmaceutical formulation, wherein said outer layer has one or more orifices that create a flow path through which fluids may pass to contact the core and dissolved drug may pass to the exterior of the device.

37. The method of claim 36, wherein the device further includes one or more semi-permeable layers disposed in said flow path, which semi-permeable layers are at least partially permeable to dissolved drug, wherein said semi-permeable layers reduce influx of proteins from ocular fluid and/or reduce the rate of release of dissolved drug from the device.

38. The method of claim 36, wherein the rate of release of drug is determined solely by the composition of the core and the total surface area of the one or more orifices relative to the total surface area of said device.

39. The method of claim 36, wherein said outer layer comprises polytetrafluoroethylene, polyfluorinated ethylenepropylene, polylactic acid, polyglycolic acid, or silicone or a mixture thereof.

40. The method of claim 36, wherein the outer layer is biodegradable.

41. The method of claim 37, wherein said semipermeable layer comprises PVA.

42. The method of claim 24, wherein the sustained release formulation is a biodegradable implant comprising said one or more drugs and a biodegradable polymer.

43. The method of claim 42, wherein said one or more drugs comprise about 50-80 weight percent of the implant.

44. A method for conducting a drug discovery business, comprising:
(A) obtaining data measuring severity of macular edema in one or more patients before and after treatment with a test compound;
(B) determining, based on the data obtained in (A), whether said severity of macular edema is reduced in said patients after treatment with said test compound;
(C) determining the suitability of further clinical development of a test compound which reduces said severity;
(D) for compounds selected for further clinical development, conducting therapeutic profiling of the test compound, or analogs thereof, for efficacy and toxicity in animals; and
(E) identifying a pharmaceutical preparation including one or more compounds identified in step (D) as having an acceptable therapeutic and/or toxicity profile.

45. The method of claim 44, further comprising licensing said compounds to a manufacturer for manufacture and sale of a pharmaceutical preparation comprising said compound.

46. A method of marketing a treatment for an ophthalmic disorder, comprising:
(A) marketing, to healthcare providers, a pharmaceutical formulation for long-term treatment of said ophthalmic disorder, which formulation includes one or more drugs that mat affect visual acuity when administered over a sustained period of time; and,
(B) providing to said healthcare providers instructions for administering said formulation, which instructions include assessing a patient's prognosis with respect to long-term visual acuity by measuring changes, if any, of macular edema as a prediction of visual acuity.

47. The method of claim 46, wherein said pharmaceutical formulation is for intraocular injection or implantation.

48. The method of claim 46, wherein said pharmaceutical formulation is a sustained-released formulation.

49. The method of claim 46, wherein said pharmaceutical formulation is provided in a sustained-release device.

50. The method of claim 46, wherein said ophthalmic disorder is: posterior uveitis, Diabetic Macular Edema (DME), Wet ARMD, or CMV retinitis.

51. The method of claim 46, wherein said pharmaceutical formulation comprises one or more of an anti-inflammatory compound, neuroprotective agent, and/or immunomodulatory compounds.

52. The method of claim 46, wherein said pharmaceutical formulation is a corticosteroid.

53. The method of claim 52, wherein said corticosteroid is: triamcinolone, dexamethasone, fluocinolone, cortisone, prednisolone, flumetholone, or derivatives thereof.

54. The method of claim 52, wherein said corticosteroid is triamcinolone acetonide (TA) or fluocinolone acetonide (FA).
